(19) United States
(12) Patent Application Publication
(10) Pub. No.: US 2006/0134217 A1
(43) Pub. Date: Jun. 22, 2006

(54) Leukotriene and integrin inhibitor combination and treatment method

(76) Inventors: Harpreet K. Sandhu, West Orange, NJ (US); David J. Valacer, San Francisco, CA (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/314,487

(22) Filed: Dec. 19, 2005

Related U.S. Application Data
(60) Provisional application No. 60/638,214, filed on Dec. 22, 2004.

(51) Int. Cl. A61K 9/24

(52) U.S. Cl. 424/472

(57) ABSTRACT

The present invention provides novel solid pharmaceutical dosage forms for oral administration comprising a therapeutically active amount of montelukast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. These novel solid pharmaceutical dosage forms are useful in the treatment or control of asthma. The present invention also provides a method for treating asthma employing the solid pharmaceutical dosage forms and a method for preparing the pharmaceutical dosage forms.

### PRIORITY TO RELATED APPLICATIONS

This application claims the benefit of U.S. Provisional Application No. 60/638,214, filed Dec. 22, 2004, which is hereby incorporated by reference in its entirety.

### FIELD OF THE INVENTION

The present invention provides novel solid pharmaceutical dosage forms for oral administration comprising a therapeutically active amount of montelukast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, and one or more pharmaceutically acceptable excipients. These novel solid pharmaceutical dosage forms are useful in the control of asthma and allergic rhinitis. The present invention also provides a method for treating asthma employing the solid pharmaceutical dosage forms and a method for preparing the pharmaceutical dosage forms.

### BACKGROUND OF THE INVENTION

Asthma

Asthma is a chronic inflammatory disorder of the airways characterized by a reduction in lung function and airway hyper-responsiveness (AHR). The airway abnormalities in asthmatics are characterized by constriction, which is the tightening of the smooth muscles surrounding the airways, and inflammation, which is the swelling and irritation of the airways and mucus plugging of small airways caused by mucus hypersecretion. Constriction, plugging and mucosal inflammation contribute to obstruction of airflow, which results in symptoms such as wheezing, coughing, chest tightness, and shortness of breath.

Airway inflammation is a hallmark of asthma. Several studies have documented an association between the numbers of eosinophils and activated lymphocytes in the airways and clinical indices of disease severity. Eosinophils are thought to be important effectors involved in bronchial mucosal damage by the release of cationic proteins, reactive oxygen species, and proinflammatory and profibrotic mediators. Much emphasis has been placed on CD4+T helper type 2 (Th2) cells as central promulgators of this inflammatory process. These Th2 lymphocytes are believed to orchestrate the events leading to the development of allergic airway responses mainly through the production of Th2-type mediators, which in turn promote the eosinophil-rich infiltrate that distinguishes asthmatic airway inflammation. Although there are available therapies focused on reducing this chronic inflammatory process in asthma, no currently available treatment has been shown to eliminate all features of the disease as a singularly effective treatment. Significant unmet medical needs remain in asthma management for patients with moderate to severe disease.

Early treatment for asthma is focused on relief of the smooth muscle contraction that leads to bronchoconstriction. A variety of medications have been used to provide quick relief and/or prevent bronchoconstriction and the resultant symptoms, e.g., wheeze, cough, exercise intolerance, and/or shortness of breath. Widely used relievers of bronchoconstriction include inhaled short-acting beta-adrenoceptor agonists such as salbutamol and albuterol, and their long acting inhaled counterparts, salmeterol and fomoterol. In addition to these inhaled beta-adrenoceptor agonists, there are controller medications that reduce airway inflammation through daily administration on a long-term basis. Inhaled corticosteroids (ICS) are the most potent and effective anti-inflammatory medications and are the first line of therapy for asthma patients. After a decade of widespread use of inhaled corticosteroids therapy, several respiratory health organizations have produced survey data which concludes that a majority of moderate to severe asthma patients do not enjoy complete and optimal control of their symptoms as defined by the widely accepted GINA/NIH (Global Initiative For Asthma/National Institutes of Health) guideline-based treatment goals. Even with higher doses of inhaled corticosteroids these patients are usually treated with multiple anti-inflammatory drugs in order to attain better levels of disease control and quality of life. Moreover, the deleterious side effects of these higher doses of inhaled corticosteroids given long-term often outweigh the clinical benefits for some patients. For this reason, the search for better complementary anti-inflammatory treatments that can spare patient exposure to higher doses of Inhaled corticosteroids has been widely advocated to provide better asthma control and prevent progression of the disease.

Role of Eosinophils and T Cells in In Asthma

The role of eosinophils in asthma is described in detail in Busse, W .W. et al., N. Engl. J. Med. 2001; 344-350, which disclosure is incorporated herein by reference. Inhaled antigens activate mast cells and Th2 cells in the airway, which in turn induce the production of mediators of inflammation such as histamine, leukotrienes and chemokines, including interleukin-4 and interleukin-5. Interleukin-5 in the bone marrow causes terminal differentiation of eosinophils. Circulating eosinophils enter the area of allergic inflammation and begin migrating to the lung by rolling, through interactions with selectins, and eventually adhering to endothelium through the binding of integrins to members of the immunoglobulin superfamily of adhesion proteins: vascular-cell adhesion molecule 1 (VCAM-1) and intercellular adhesion molecule 1 (ICAM-1). As the eosinophils enter the matrix of the airway through the influence of various chemokines and cytokines (such as MCP-1, monocyte chemotactic protein, and MIP-1 (, macrophage inflammatory protein), their survival is prolonged by interleukin-5 and granulocyte-macrophage colony-stimulating factor (GM-CSF). On activation, the eosinophil releases inflammatory mediators such as leukotrienes and granule proteins to injure airway tissues. In addition, eosinophils can generate granulocyte-macrophage colony-stimulating factor to prolong and potentiate their survival.

The presence of activated CD4 Th2 cells is also a hallmark feature of asthma in particular of chromic asthma. The persistence of Th2 cells may be the result of an increased recruitment and a prolonged survival in the airway tissue interstium (Cohn L, Elias JA, Chupp GL. Annual Review of Immunology. 2004. 22 (1): 789-815). As with eosinophils, Th2 cells enter the airways from the vascular through interaction of adhesion molecules with the vascular endothelium. Once in the tissue, these cells encounter antigen presenting cells, such as dendritic cells, where they proliferate. This costimulatory response as well as the resistance to apoptosis may be mediated by alpha4-VCAM-1 interactions.

Early and Late Phase Reactions to Allergens

In controlled inhaled allergen challenge experiments, sensitized asthmatic patients develop an early-phase allergic response (EAR) that occurs within minutes and most often resolves spontaneously within 30 to 60 minutes. This early-phase allergic response results primarily from the release of preformed pro-inflammatory mediators such as histamine as well as the de novo generation of leukotrienes C₄, D₄, and E₄ by bronchial mast cells. These mediators induce smooth muscle contraction, mucus secretion, and vasodilatation. Inflammatory mediators also induce microvascular leakage of plasma proteins, causing edematous swelling of the airway walls and a narrowing of the airway lumen.

This early-phase allergic response is usually followed by a second phase of airflow obstruction, termed the late-phase allergic response (LAR), which occurs 6 to 10 hours later. The late-phase allergic response develops as a result of cytokines and chemokines generated by resident cells of the lung (mast cells, macrophages, and epithelial cells) and recruited inflammatory cells (T lymphocytes and eosinophils). The T lymphocytes involved in this process are of the Th2 type and are found in a wide variety of hypersensitivity reactions including allergic rhinitis as well as asthma. Th2 cells produce interleukins, which have pronounced effects on inflammatory cells, particularly eosinophils. Circulating eosinophils migrate into the airway. Upon activation, eosinophils release inflammatory mediators such as leukotrienes, and granule proteins such as major basic protein to injure airway tissues. Features of the late-phase allergic response include bronchospasm, escalating inflammation, mucus hypersecretion and airway wall edema. Swelling of the airway wall also leads to a loss of elasticity, which further contributes to airflow limitation. An additional consequence of the late-phase allergic response is an increase in airway hyper-responsiveness, which reinforces and perpetuates the asthmatic response.

The Integrins

The integrins constitute a large class of heterodimeric, cell surface molecules consisting of α and β chains, each of which has a large extracellular domain and a short cytoplasmic tail. There are at least 14 different a chains and 8 β chains known, which combine in a restricted manner depending on cell type to give approximately 23 members of the integrin family, each of which binds specific peptide ligands. Integrins mediate a variety of cell functions including adhesion, migration, activation and survival. Lymphocytes and leukocytes with the exception of neutrophils constitutively express the integrin VLA4 (α₄β₁, very late activating antigen4, CD-49d/CD-29) and are capable of expressing the closely related integrin, α₄β₇.

The α₄β₇ and α₄β₇ integrins mediate cell-cell adhesion to the immunoglobulin superfamily member, vascular cell adhesion molecule-1 (VCAM-1), and cell-matrix adhesion to fibronectin. In addition, α₄β₇ also binds mucosal addressin cell adhesion molecule-1 (MadCAM-1). VCAM-1 regulates leukocyte migration from the blood into tissues. VCAM-1 expression is induced on endothelial cells during inflammatory responses such as that seen in asthma.

In asthma, there is increased expression of α₄β₁ and α₄β₇ integrins on all mononuclear leukocytes (including Th2 cells), eosinophils, basophils, and mast cells. The selective and increased expression of the α₄ integrins only on those cells involved in the inflammatory cascade in asthma would suggest that it is possible to target the underlying disease process without compromising normal host-defense responses.

In vivo studies with monoclonal antibodies (MoAbs) to the α₄ chain of α₄β₁ and α₄β₇ in several animal models of asthma demonstrate that α₄ integrins play a key role in eosinophil and T cell recruitment, activation, and survival leading to a significant reduction of airway inflammation. Furthermore, antibodies directed against VLA-4 block eosinophil accumulation, hyper-reactivity, and inflammation in mouse, rat and guinea pig models of allergic asthma. More recently the peptide VLA-4 antagonist, Bio1211, was shown to block late phase airway response as well as to attenuate carbacol induced airway hyper-responsiveness in a sheep model of allergic asthma. Lastly, VCAM-deficient mice show no signs of airway inflammation.

R411

R411 (N-(2-Chloro-6-methylbenzoyl)-4-[(2,6-dichlorobenzoyl)amino]-L-phenylalanine-2-(diethylamino)ethyl ester) is an ester pro-drug of the active moiety, N-(2-chloro-6-methylbenzoyl)-4-[(2,6-dichlorobenzoyl)amino]-L-phenylalanine. R411 has the following chemical structure:

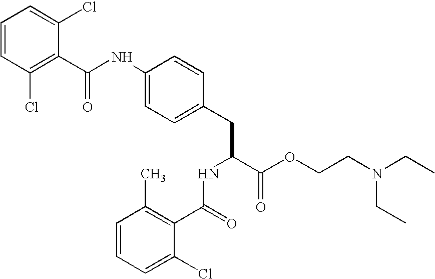

R411 inhibits the binding of α₄/β₁ to vascular cell adhesion molecule (VCAM-1) and α₄/β₇ to MadCAM-1 by binding to. R411 is disclosed in U.S. Pat. No. 6,229,011, which disclosure is incorporated by reference herein.

R411 will only modulate immune responses mediated by α₄-integrins and, therefore in asthma, selectively target only those inflammatory cells involved in the pathogenesis of the disease: Th2 cells, eosinophils, and mast cells. The expression of α₄-integrins on these cells is increased in asthma mediating their recruitment, activation, retention, and survival in the airways. The alpha4 integrins appear not to be involved in cellular immunity and other humoral host defense responses. Therefore R411 would be expected to selectively target the inflammatory response in asthma without compromising normal host-defense.

R411 binds with high affinity and slow dissociation from the activated α₄ ligand. In contrast, in vitro binding affinity is lower and dissociation is more rapid when the receptor is not activated. While Bio1211 is specific for α₄/β₁ integrin, R411 is effective against both α₄/β₁ and α₄/β₇ integrins.

R411 can attenuate airway hyper-responsiveness; reduce edema; reduce smooth muscle hypertrophy/mucus gland hyperplasia; block trafficking of leukocytes to airways; increase peripheral blood lymphocytes and eosinophils; modulate Th2 cytokine production; block costimulatory signals for T cells and eosinophils; and inhibit eosinophil survival. In our experimental studies, R411 was observed to block the migration of key inflammatory cells from the blood into the lungs.

Many α-lntegrin inhibitors having various inhibitory selectivity patterns have been disclosed; see e.g.: U.S. Pat. Nos. 6,380,387; 6,388,084; 6,420,600; 6,423,728; 6,455,550; and 6,734,311.

Activity of Cysteinyl Leukotrienes

The cysteinyl leukotrienes (LTC₄, LTD₄, LTE₄) are products of arachidonic acid metabolism and are released from various cells, including mast cells and eosinophils. These eicosanoids bind to cysteinyl leukotriene (CysLT) receptors. The CysLT type-1 (CysLT₁) receptor is found in the human airway (including airway smooth muscle cells and airway macrophages) and on other pro-inflammatory cells (including eosinophils and certain myeloid stem cells). CysLTs have been shown to be important mediators in the pathophysiology of asthma and allergic rhinitis. In asthma, leukotriene-mediated effects include airway edema, smooth muscle contraction, and altered cellular activity associated with the inflammatory process.

Cys-LTs have been well recognized in the past for their powerful bronchoconstricting effects and for their role in asthma exacerbations. Cys-LTs are present in human bronchoalveolar lavage (BAL) fluid from subjects after allergen challenge. Elevated levels of LTE₄ were seen in urine samples collected from patients visiting the emergency room for treatment of asthma exacerbations. Orally administered CysLT₁ receptor antagonists also attenuate bronchoconstrictive responses to challenges with exercise and cold air. In addition, the involvement of cys-LTs in the afferent limb of adaptive immunity (particularly the induction of Th2 responses in the lung via effects on dendritic cells and cytokine generation), the recruitment and/or activation of effector cells (especially eosinophils and mast cells), inflammation, and fibrosis have all been supported by animal models and await validation in humans.

Taken together, these observations confirm the involvement of CysLTs (LT C4, D4, E4) in the development of airflow obstruction in both experimentally induced and naturally occurring asthma in humans, and with a prominent role for the CysLT₁ receptor in asthma exacerbations.

Montelukast

Montelukast sodium, the active ingredient in SINGULAIR®, is a selective and once daily, orally administered leukotriene receptor antagonist that inhibits the cysteinyl leukotriene CysLT₁ receptor. Montelukast sodium is [R-(E)]-1-[[[1-[3-[2-(7-chloro-2-quinolinyl)ethenyl] phenyl]-3-[2-(1-hydroxy-1-methylethyl)phenyl]propyl]thio]methyl]cyclopropaneacetic acid, monosodium salt and has the following chemical structure:

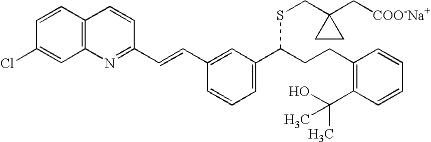

According to the Orange Book, montelukast sodium is disclosed in U.S. Pat. No. 5,565,473, which disclosure is incorporated by reference herein.

Each 10-mg film-coated SINGULAIR® tablet contains 10.4 mg montelukast sodium, which is equivalent to 10 mg of montelukast, and the following inactive ingredients: microcrystalline cellulose, lactose monohydrate, croscarmellose sodium, hydroxypropyl cellulose, and magnesium stearate. The film coating consists of hydroxypropyl methylcellulose, hydroxypropyl cellulose, titanium dioxide, red ferric oxide, yellow ferric oxide, and carnauba wax.

Montelukast has a single known mechanism of action as a selective antagonist of the CystLT1 receptor. Blockade of the CysLT1 results clinically in mild bronchodilator effects with an effect demonstrable by FEV1 (Forced Expiratory Volume in 1 second) measurements that begins within hours of first dose and a maximal effect within 2-4 weeks. This mild bronchodilatory effect has been shown to be sustained over 12 weeks of treatment. Intravenous administration of montelukast, substantially increased measures of airflow compared with placebo in a group of patients presenting to the emergency room with acute asthma who also received standard treatment with bronchodilators and glucocorticoids.

Additional in vitro and/or in vivo effects of montelukast include blocking induction of cytokine generation by eosinophils and MCs resulting in a reduction in circulating eosinophils in peripheral blood and in BAL fluid; reducing eosinophil recruitment in allergic rhinitis with efficacy in allergic rhinitis; reducing activated T cells; reducing Th2 cytokine production; modulating Beta2 integrin expression; and reducing edema/mucus hypersecretion. (SINGULAIR® package insert)

### BRIEF DESCRIPTION OF THE FIGURES

FIG. 1 is a graph illustrating the efficacy of montelukast for the chronic treatment of asthma in adults employing the primary endpoint, FEV₁, expressed as mean percent change from baseline.

FIG. 2 is a graph illustrating the additive effect of R411 on moderate dose inhaled corticosteroids in large airway flow rates as measured by FEV₁.

FIG. 3 is a graph illustrating the additive effect of R411 on moderate dose inhaled corticosteroids in large airway flow rates as measured by FEF25-75.

FIG. 4 is a graph illustrating the effect of R411 on small airway flow rates as measured by FEF25-75 when administered as monotherapy to asthmatic patients.

FIG. 5 is a picture illustrating the preferred novel solid oral dosage forms of the invention, specifically a bilayer tablet, a sandwich tablet, a tablet containing coated microbeads, and a film coated tablet.

FIG. 6 is a bar graph showing that the oral administration of R411 attenuates airway inflammation in the atopic primate.

FIG. 7 is a bar graph comparing the effects of R411, fluticasone, and montelukast on neutrophils, eosinophils, and lymphocytes in the primate.

### SUMMARY OF THE INVENTION

The present invention provides a solid pharmaceutical dosage form for oral administration comprising a therapeutically active amount of montelukast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

The present invention also provides a method for treating asthma comprising administering to a subject, in need thereof, a solid pharmaceutical dosage form for oral administration comprising a therapeutically active amount of montelukast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

The present invention further provides a method for preparing a solid pharmaceutical dosage form for oral administration comprising admixing a therapeutically active amount of montelukast, or a pharmaceutically acceptable salt thereof, a therapeutically effective amount of N-(2-chloro-6-methylbenzoyl)-4-[(2,6-dichlorobenzoyl)amino ]-L-phenylalanine-2-(diethylamino)ethyl ester, or a pharmaceutically acceptable thereof, and one or more pharmaceutically acceptable excipients.

### DETAILED DESCRIPTION OF THE INVENTION

The present invention provides solid pharmaceutical dosage forms for oral administration comprising a therapeutically active amount of montelukast, or a pharmaceutically acceptable salt thereof, a therapeutically effective amount of R411, or a pharmaceutically acceptable thereof, and one or more pharmaceutically acceptable excipients. In a preferred embodiment, the dosage form comprises a combination of two discrete pre-formulated pharmaceutical compositions. The first composition comprises a therapeutically active amount of montelukast, or a pharmaceutically acceptable salt thereof, formulated with one or more pharmaceutically acceptable excipients. The second composition comprises a therapeutically effective amount of R411, or a pharmaceutically acceptable salt thereof, formulated with one or more pharmaceutically acceptable excipients. In a more preferred embodiment, the dosage form comprises two discrete regions. The first region comprises a therapeutically effective amount of montelukast, or a pharmaceutically acceptable salt thereof. The second region comprises a therapeutically effective amount of R411, or a pharmaceutically acceptable salt thereof. These oral dosage forms are useful in the treatment or control of asthma and allergic rhinitis.

The pharmaceutical dosage forms of the present invention provide two compounds for treating asthma that operate by complementary mechanisms of action. Montelukast affects the early-phase allergic response through its direct blockade of cysLTs binding to its receptor. R411 inhibits eosinophil and Th2 cell excitation and survival, and inhibits eosinophil migration from blood to pulmonary tissues. The combination of the two compounds in the pharmaceutical dosage forms therefore provides a therapeutic treatment that has the combined effect of reducing circulating eosinophil counts and reducing eosinophil egress into pulmonary tissues thereby providing an early onset of bronchodilation as well as sustained anti-inflammatory effects. Hence administration of the pharmaceutical dosage forms of the present invention provides a means of intensifying asthma therapy while supporting good patient compliance.

Since montelukast is a weak acid and R411 is a weak base, the novel solid pharmaceutical dosage forms of the invention require specific pharmaceutical dosage formulations. Directly combining the two compounds may not achieve the desired effect since the bioavailability, solubility, or stability of one compound may be compromised by the presence of the other compound. It has been discovered that it is preferable that the two active ingredients are instead first pre-formulated separately to obtain pharmaceutically acceptable stability and bioavailability characteristics for each ingredient. The two separately pre-formulated active ingredients are then combined in an appropriate solid dosage composition for oral administration. Particularly preferred solid dosage forms are those in which the separately pre-formulated ingredients are combined in a dosage form having separate discrete regions for the two pre-formulated ingredients such as by discrete layers, encapsulations, and the like. Examples of such dosage forms include, but are not limited to, a bilayer tablet, a sandwich tablet, a tablet having coated microbeads, or a film coated tablet.

The pharmaceutical dosage forms may also be formulated to provide a chronobiological synergy of the two compounds. R411 is a weak base and therefore has a higher solubility in the upper part of the gastrointestinal tract, i.e., stomach and duodenum, whereas montelukast is a weak acid and has a higher solubility in the lower part of the gastrointestinal tract (small intestine and colon). To maximize the therapeutic administration of the present pharmaceutical dosage form, a sustained release or delayed release formulation of montelukast may be combined with an immediate release formulation of R411 to provide better disease management.

As used herein, the following terms have the given meanings:

“Montelukast” refers to montelukast, and pharmaceutically acceptable salts thereof.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., H. Ansel et. al., Pharmaceutical Dosage Forms and Drug Delivery Systems (6^{th} Ed. 1995) at pp. 196 and 1456-1457.

“Prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bioreversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

“R411” refers to N-(2-chloro-6-methylbenzoyl)-4-[(2,6-dichlorobenzoyl)amino]-L-phenylalanine-2-(diethylamino)ethyl ester.

“Therapeutically effective amount” means an amount of at least one compound of the invention, or a pharmaceutically acceptable salt thereof, which is effective to prevent, alleviate or ameliorate symptoms of disease or prolong the survival of the subject being treated. Determination of a therapeutically effective amount is within the skill in the art.

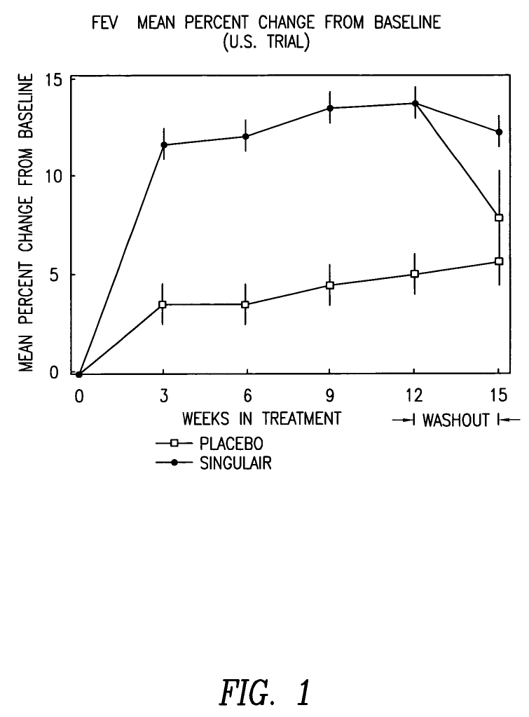

As set out above, one component in the solid pharmaceutical dosage form comprises a therapeutically effective amount of montelukast, or a pharmaceutically acceptable salt thereof. The efficacy of montelukast for the chronic treatment of asthma in adults and adolescents 15 years of age and older was demonstrated in two (U.S. and Multinational) similarly designed, randomized, 12-week, double-blind, placebo-controlled trials in 1576 patients (795 treated with montelukast, 530 treated with placebo, and 251 treated with active control). The patients studied were mild and moderate, non-smoking asthmatics who required approximately 5 puffs of inhaled (beta)-agonist per day on an “as-needed” basis. The patients had a mean baseline percent of predicted forced expiratory volume in 1 second (FEV₁) of 66% (approximate range, 40 to 90%). The co-primary endpoints in these trials were FEV, and daytime asthma symptoms. Secondary endpoints included morning peak expiratory flow rates (AM PEFR) and evening peak expiratory flow rates (PM PEFR), rescue (beta)-agonist requirements, nocturnal awakening due to asthma, and other asthma-related outcomes. In both studies after 12 weeks, a random subset of patients receiving montelukast was switched to placebo for an additional 3 weeks of double-blind treatment to evaluate for possible rebound effects. The results of the U.S. trial on the primary endpoint, FEV₁, expressed as mean percent change from baseline, are shown in FIG. 1. FEV₁ reflects the caliber and integrity of the large airways and improvements reflect bronchodilation of the larger airways. (Singulair® Package Insert, US)

One randomized, placebo-controlled, parallel-group trial (n=226) enrolled stable asthmatic adults with a mean FEV₁ of approximately 84% who were previously maintained on various inhaled corticosteroids (delivered by metered-dose aerosol or dry powder inhalers). The types of inhaled corticosteroids and their mean baseline requirements included beclomethasone dipropionate (mean dose, 1203 mcg/day), triamcinolone acetonide (mean dose, 2004 mcg/day), flunisolide (mean dose, 1971 mcg/day), fluticasone propionate (mean dose, 1083 mcg/day), or budesonide (mean dose, 1192 mcg/day). Some of these inhaled corticosteroids were non-U.S.-approved formulations, and doses expressed may not be ex-actuator. The pre-study inhaled corticosteroid requirements were reduced by approximately 37% during a 5- to 7-week placebo run-in period designed to titrate patients toward their lowest effective inhaled corticosteroid dose. Treatment with montelukast resulted in a further 47% reduction in mean inhaled corticosteroid dose compared with a mean reduction of 30% in the placebo group over the 12-week active treatment period (p</=0.05). Approximately 40% of the montelukast-treated patients and 29% of the placebo-treated patients were successfully tapered off inhaled corticosteroids, remaining free of inhaled corticosteroids therapy at the conclusion of the study (p=NS). It is not known whether the results of this study can be generalized to apply to asthmatics who require higher doses of inhaled corticosteroids or systemic corticosteroids. Similar studies have not been performed in asthmatic patients requiring higher doses of inhaled and/or systemic corticosteroid therapy but cumulative clinical experience suggests that montelukast's steroid sparing effects are not as great in more severe patients.

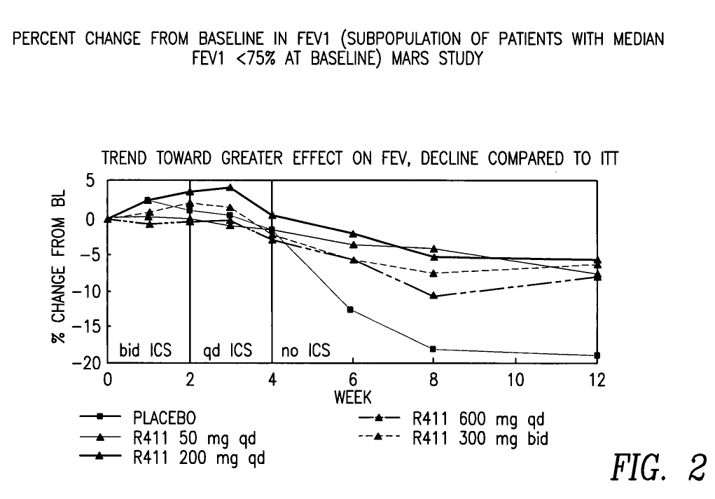

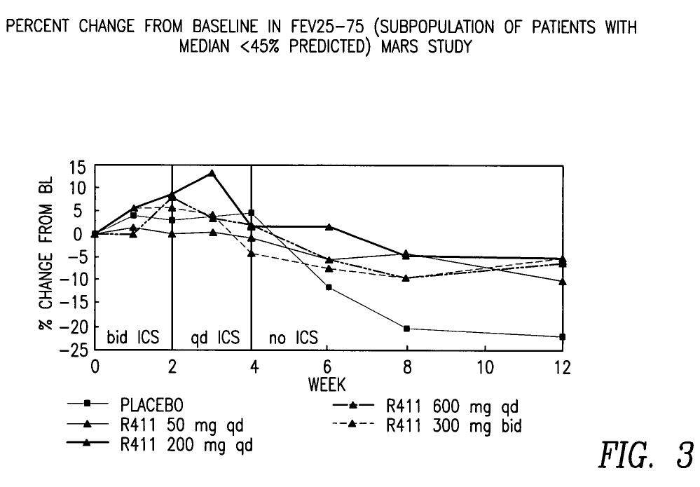

As set out above, a second component in the solid pharmaceutical dosage form comprises a therapeutically effective amount of R411 (N-(2-chloro-6-methylbenzoyl)-4-[(2,6-dichlorobenzoyl)amino]-L-phenylalanine-2-(diethylamino)ethyl ester), or a pharmaceutically acceptable salt thereof. In Phase II studies, in a subpopulation of patients with less well controlled asthma, R411 demonstrated an additive effect to moderate dose inhaled corticosteroids in large airway flow rates as measured by FEV₁ (FIG. 2) and small airway flow rates measured by FEF25-75 (FIG. 3). The MARS study illustrated in FIG. 3 was designed to evaluate the safety and efficacy of R411 over a 12 week treatment period in 350 persistent asthmatics being treated with a stable dose of low to medium inhaled corticosteroids and inhaled short acting β12-agonist. Patients were randomized to one of five cohorts: 50, 200, 600 mg once daily (QD) or 300 mg twice daily (BID) R411, or placebo (n=70/group). After a 2-week placebo run-in period and subsequent 2-week add-on period (R411 or placebo), morning inhaled corticosteroids were removed. Two weeks later, evening inhaled corticosteroids were removed, and patients remained in the treatment period for an additional 8 weeks. The primary endpoint in the study was the percentage change in FEV1 from baseline, and secondary endpoint included PEFR, asthma exacerbations, β2-agonist use, asthma control questionnaire, asthma symptom scores, nocturnal awakenings, FEF25-75 and rate of asthma treatment failures.

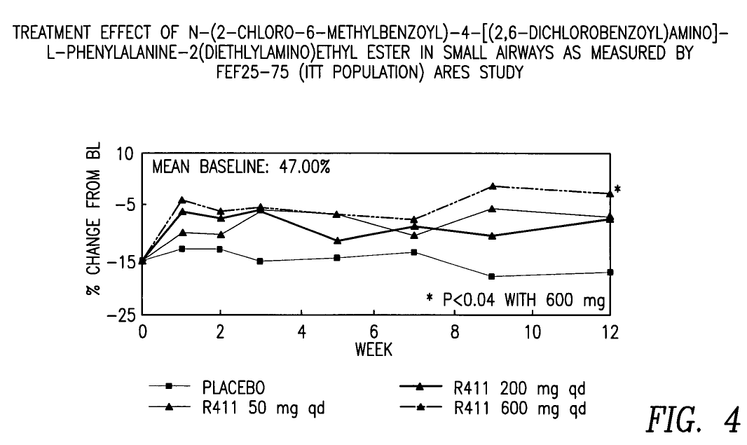

A significant effect on small airway flow rates as measured by FEF25-75 was seen with R411 even when administered as monotherapy to a milder population (ARES study) of asthmatic patients (FIG. 4). The ARES study illustrated in FIG. 4 was designed to evaluate the safety and efficacy of monotherapy R411 over a 12 week treatment period in 480 mild/moderate asthmatics not treated with inhaled corticosteroids. Patients were randomized to one of four cohorts: 50, 200, 600 mg QD R411, or placebo (n=120/group). The primary endpoint in the study was change in FEV1 from baseline, and secondary endpoints included PEFR, asthma exacerbations, β2-agonist use, asthma control questionnaire, asthma symptom scores, and nocturnal awakenings. Small airway inflammation represents a clinically significant component of moderate to severe asthma that has generally not been adequately controlled by conventional inhaled corticosteroid therapies. Therefore, R411 represents a novel opportunity to further address important unmet needs in more severe asthmatic populations.

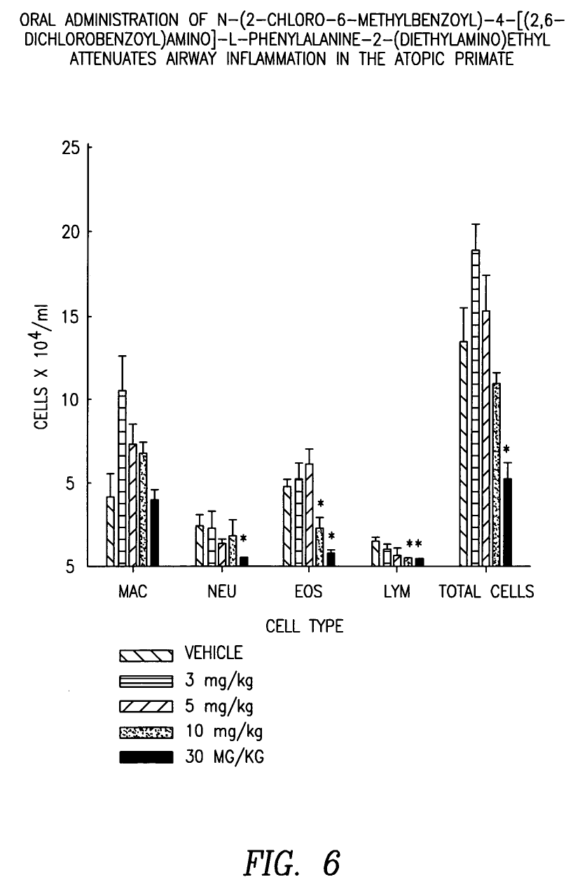

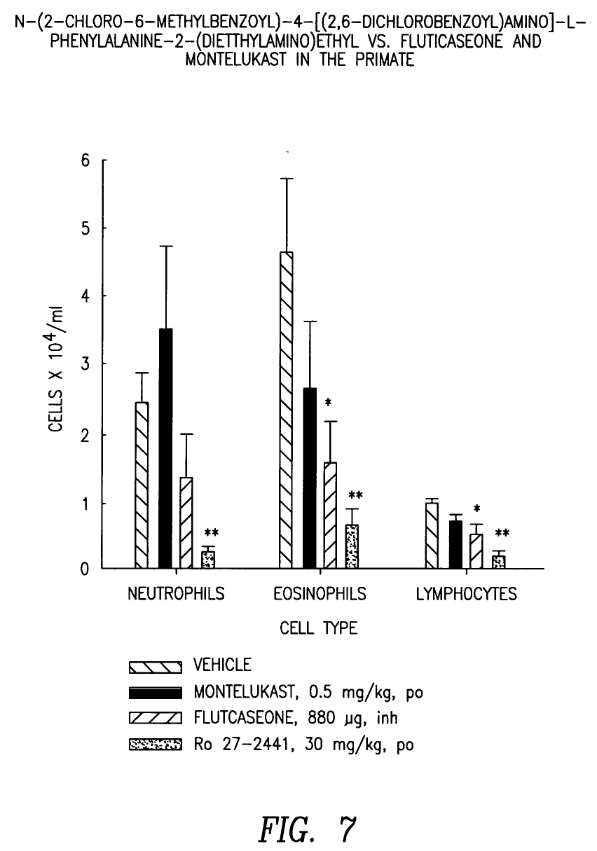

FIG. 6 is a bar graph showing that the oral administration of R411 attenuates airway inflammation in the atopic primate. FIG. 7 is a bar graph comparing the effect of R411, fluticasone, and montelukast on neutrophils, eosinophils, and lymphocytes in the primate. With regard to CD4Th2 (helper) cells, montelukast reduces levels of Th2 cytokines; R411 inhibits migration from blood to pulmonary tissues; R411 inhibits T cell costimulation/proliferation; and R411 selectively modulates Th2 cytokines levels.

Improvement on Asthma and Allergy Symptoms (Allergic Asthma vs. Non-Allergic Asthma)

In adult patients, montelukast reduced “as-needed” (beta)-agonist use by 26.1% from baseline compared with 4.6% for placebo. In patients with nocturnal awakenings of at least 2 nights per week, montelukast reduced the nocturnal awakenings by 34% from baseline, compared with 15% for placebo (combined analysis). (Singulair® Package Insert, US)

R411 also has positive effects on symptoms of asthma. ARES study evaluated the safety and efficacy of monotherapy with R411 over a 12-week treatment period in 479 mild/moderate asthmatics not treated with inhaled corticosteroids. Patients were randomized to one of four cohorts: 50, 200, 600 mg once daily R411, or placebo. Statistically significant improvements with R411 were achieved in reducing rescue albuterol use, decrease in daytime asthma and nocturnal symptom score. Improvement in Asthma Control Questionnaire Scores and Asthma Quality-of-Life were also observed when compared to placebo. Although the study was not powered to detect significant differences in asthma exacerbations, a 26% reduction was observed with the two highest doses of 200 and 600 mg. The results are set out in the Table below.

Secondary Efficacy Endpoints in the ITT Population when
R411 is Administered as Monotherapy (ARES Study)

| | Change from Baseline ITT Population | | |
|---|---|---|---|
| | (Median FEV1 74.75% at Baseline) Table | | |
| | Secondary Efficacy Endpoints in the ITT | | |
| | Population when R411 is Given as | | |
| | Monotherapy (ARES Study) | | |
| | Placebo | 200 mg | 600 mg |
| | (N = 117)/ | (N = 117)/ | (N = 119)/ |
| | [Mean BV] | [Mean BV]) | [Mean BV] |
| Rescue β 2-agonist use | 0.1/[2.98] | −0.36/[3.04] | −0.41*/[3.11] |
| (puffs/d) | | | |
| Nocturnal awakenings | 0.06/[0.54] | −0.15*/[0.61] | −0.12*/[0.58] |
| (scores) | | | |
| Morning asthma | −0.13 [1.61] | −0.34* [1.55] | −0.33* [1.57] |
| symptoms | | | |
| Asthma control | −0.07 [2.00] | −0.29 [2.08] | −0.23 [2.00] |
| Questionnaire (Total | | | |
| Score) | | | |
| % Asthma exacerbation | 32.50 | 25.6 | 26.10 |

*p < 0.05, before adjustments for multiple comparisons;
BV = baseline value for the group.

Solid Oral Dosage Forms Comprising Montelukast and R411

In accordance with the present invention, solid pharmaceutical dosage forms for oral administration are provided comprising a therapeutically active amount of montelukast, or a pharmaceutically acceptable salt thereof, a therapeutically effective amount of R411, or a pharmaceutically acceptable thereof, and one or more pharmaceutically acceptable excipients. In a preferred embodiment, the dosage form comprises a combination of two discrete pre-formulated pharmaceutical compositions, the first composition comprising montelukast and the second composition comprising R411. In a more preferred embodiment, the dosage form comprises two discrete regions, the first region comprising montelukast and the second region comprising R411. These once daily oral dosage forms are useful in the treatment or control of asthma.

Without intending to limit the invention as claimed herein to any particular theory, the pharmaceutical dosage forms of the present invention are believed to provide an improved efficacy profile in the treatment of asthma by virtue of complementary mechanisms of action. Montelukast affects the early-phase asthmatic response through its direct blockade of cysLTs binding to its receptor. In patients 2 years and older with asthma who received montelukast, a decrease in mean peripheral blood eosinophil counts ranging from 9% to 15% was noted, compared with placebo, over the double-blind treatment periods (Singulair Package Insert). The specific mechanism of action of R411 suggests that it's greatest effect will be on the late-phase allergic response in animal and human challenge studies characterized by its effect on eosinophils. R411 inhibits eosinophil excitation and survival, inhibits eosinophil migration from blood to pulmonary tissues, and may promote apoptosis of tissue eosinophils though integrin blockade. Administration of a solid oral dosage form containing both montelukast and R411 therefore provide a therapeutic treatment having the combined effects of reducing circulating eosinophil counts and reducing eosinophil egress into pulmonary tissues. Administration of the dosage form containing both compounds of the present invention will provide an improved anti-inflammatory effect than that achieved by administration of either drug alone by virtue of their complementary modes of action.

The therapeutically effective amount or dosage of montelukast and R411 according to this invention can vary within wide limits and may be determined in a manner known in the art. Such dosage will be adjusted to the individual requirements in each particular case including the specific compound(s) being administered, the condition being treated, as well as the patient being treated. In general, in the case of oral administration of montelukast, or pharmaceutically acceptable salts thereof, to adult humans weighing approximately 70 Kg, montelukast will be present in a daily dosage ranging from about 2 mg to about 10 mg. In general, in the case of oral administration of R411, or pharmaceutically acceptable salts thereof, to adult humans weighing approximately 70 Kg, R411 will be present in a daily dosage ranging from about 50 mg to about 400 mg, and preferably from about 50 to about 200 mg.

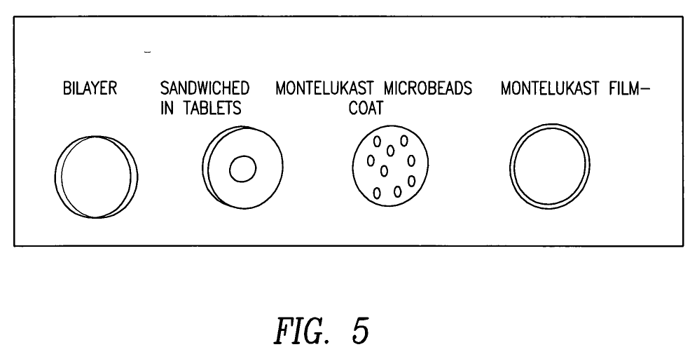

As set out above, directly combining montelukast and R411 may not achieve the desired effect since the bioavailability, solubility, or stability of one compound may be compromised by the presence of the other compound. It is preferable that the two active ingredients are instead first pre-formulated separately to obtain pharmaceutically acceptable stability and bioavailability characteristics for each ingredient. The two separately pre-formulated active ingredients are then combined in an appropriate solid dosage composition for oral administration. Particularly preferred solid dosage forms are those in which the separately pre-formulated ingredients are combined in a dosage form having separate discrete regions for the two pre-formulated ingredients such as by discrete layers, encapsulations, and the like. Examples of such dosage forms include, but are not limited to, a bilayer tablet, a sandwich tablet, a tablet having coated microbeads, or a film coated tablet. (FIG. 5)

In general, bilayer tablets may be formulated by utilizing twin hopper compression machines. The granulates of each compound may be prepared individually using pharmaceutically acceptable excipients such as lactose, sucrose, microcrystalline cellulose, stearic acid, hydroxypropylmethylcellulose, polyvinylpyrrolidone, crospovidone, croscarmelose sodium, sodium starch glycolate, dicalcium phosphate, mannitol, sorbitol, silicified microcrystalline cellulose, talc, colloidal silica, stearic acid, or magnesium stearate. The individual granulates can then be compressed together into one unit.

In a specific embodiment, the bilayer tablet may comprise: (a) a first layer comprising montelukast present in an amount from about 2 mg to about 10 mg; and (b) a second layer comprising R411 present in an amount from about 50 mg to about 400 mg.

In general, sandwich tablets (or tablets inside tablets) can be prepared by sandwiching a tablet of montelukast unit into the granulates of R411 using twin hopper compression machines. The tablet of montelukast is prepared by using standard excipients described above and the granulates of R411 are prepared by conventional granulation techniques using pharmaceutically acceptable excipients.

In a specific embodiment, the pharmaceutical dosage form is a sandwich tablet comprising: (a) an inner core layer comprising montelukast present in an amount from about 2 mg to about 10 mg; and (b) an outer surrounding layer comprising R411 present in an amount from about 50 mg to about 400 mg.

In another specific embodiment, the pharmaceutical dosage form is a sandwich tablet comprising: (a) an inner core layer comprising R411 present in an amount from about 50 mg to about 400 mg; and (b) an outer surrounding layer comprising montelukast present in an amount from about 2 mg to about 10 mg.

In general, tablets having coated microbeads can be prepared by formulating one of the components, such as montelukast, using either granulation or granulation followed by extrusion-merumerization techniques and coating the component with pharmaceutically acceptable polymers such as hypromellose, ethylcellulose, hydroxypropylcellulose, polyvinylalcohol, and/or aminomethylmethacrylate in fluid bed or coating pans in such a proportion that coating provides enough barrier to separate the two active components but does not affect the dissolution behavior of the coated product. The coated microbeads of montelukast can then be mixed with R411 granulates prepared using conventional methods. These mixed granulations can be used to prepare tablets, capsules, or suspensions, or can be dispersed in an oily matrix. Separating the granulation process and further coating of those granulates help provide the barrier required to keep the two components separate while not affecting the dissolution behavior thus assuring the desired pharmacokinetic exposures.

In a specific embodiment, the pharmaceutical dosage form is a tablet having coated microbeads comprising: (a) a tablet comprising R411 present in an amount from about 50 mg to about 400 mg; and (b) coated microbeads dispersed throughout the tablet comprising montelukast present in an amount from about 2 mg to about 10 mg.

In another specific embodiment, the pharmaceutical dosage form is a tablet having coated microbeads comprising: (a) a tablet comprising montelukast present in an amount from about 2 mg to about 10 mg; and (b) coated microbeads dispersed throughout the tablet comprising R411 present in an amount from about 50 mg to about 400 mg.

In general, film-coated tablets can be prepared by incorporating montelukast in a film-coating layer. Tablets of R411 are prepared by conventional manufacturing processes such as granulation, milling, blending, lubricating, and compressing. The required dose of montelukast is dissolved in a coating dispersion usually consisting of film forming agents such as hypromellose (hydroxypropyl methylcellulose), polyvinyl alcohol, starch or ethylcellulose along with a gliding agent such as talc, colorant and plasticizer (triacetin, dibutylsebacate, polyethylene glycol) dispersed in water. The required amount of montelukast film coating is then applied over the R411 kernel tablet either in a pan coater or fluidbed coater to deposit the specific amount of montelukast onto the R411 kernels.

In a specific embodiment, the pharmaceutical dosage form is a film coated tablet comprising: (a) a tablet comprising R411 present in an amount from about 50 mg to about 400 mg; and (b) a film coating covering the tablet comprising montelukast present in an amount from about 2 mg to about 10 mg.

In another specific embodiment, the pharmaceutical dosage form is a film coated tablet comprising: (a) a tablet comprising montelukast present in an amount from about 2 mg to about 10 mg; and (b) a film coating covering the tablet comprising R411 present in an amount from about 50 mg to about 400 mg.

The process of granulation consists of granulation with water or an appropriate solvent in a low or high shear granulator, fluid bed dryer, dry granulation with roller compaction or slugging or melt granulation using polyethylene glycols, phospholipids, poloxamers, monoglycerides, diglycerides and triglycerides, fatty acids, polyglycolized ester such as Gelucires, Vitamin E TPGS or by melt extrusion using thermosetting polymers such as polyvinylpyrrolidone, poloxamers, polyethylene glycol, ethyl cellulose, stearic acid, glyceryl monostearate, glyceryl behenate, and/or sucrose diesters. In order to manufacture the oral suspension, transdermal patches, these granulates in the desired proportion are dispersed in pharmaceutical bases consisting of excipients such as polyethylene glycols, surfactants Cremophor EL, Cremophor RH40, Solutol HS15, Gelucires 44/14, 50/15, 39/01, 33/01, polysorbates, spans, sodium dodecyl sulfate can be added to further improve the absorption process.

The pharmaceutical dosage forms may also be formulated to provide a chronobiological synergy of the two compounds. R411 is a weak base and therefore has a higher solubility in the upper part of the gastrointestinal tract, i.e., stomach and duodenum, whereas montelukast is a weak acid and has a higher solubility in the lower part of the gastrointestinal tract (small intestine and colon). To maximize the therapeutic administration of the present pharmaceutical dosage form, a sustained release or delayed release formulation of montelukast may be combined with an immediate release formulation of R411 to provide better disease management. The delayed release of montelukast is achieved by either by diffusion-controlled membrane such as ethylcellulose or non-ionic polymethylmethacrylates (RL, RS and NE Eudragits) or the enteric coating by anionic methylmethacrylates (Eudragit L and S), polyvinyl acetate phthalate, acetylysuccinate.

In another embodiment, the present invention provides a method for treating asthma comprising administering to a subject, in need thereof, a solid pharmaceutical dosage form for oral administration comprising a therapeutically active amount of montelukast, or a pharmaceutically acceptable salt thereof, a therapeutically effective amount of R411, or a pharmaceutically acceptable thereof, and one or more pharmaceutically acceptable excipients. Preferably, the dosage form comprises a combination of two discrete pre-formulated pharmaceutical compositions, the first composition comprising montelukast and the second composition comprising R411. More preferably, the dosage form comprises two discrete regions, the first region comprising montelukast and the second region comprising R411.

Preferably, montelukast is present in an amount from about 2 mg to about 10 mg and R411 is present in an amount from about 50 mg to about 400 mg. Preferably, the dosage form is selected from the group consisting of bilayer tablet, a sandwich tablet, a tablet having coated microbeads, and a film coated tablet.

In yet another embodiment, the present invention provides a method for preparing a solid pharmaceutical dosage form for oral administration comprising admixing a therapeutically active amount of montelukast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

Preferably, the method further comprises: (A) providing a first pre-formulated pharmaceutical composition comprising a therapeutically active amount of montelukast, or a pharmaceutically acceptable salt thereof, formulated with one or more pharmaceutically acceptable excipients; (B) providing a second pre-formulated pharmaceutical composition comprising a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, formulated with one or more pharmaceutically acceptable excipients; and (C) combining the first and second solid compositions to form the pharmaceutical dosage form.

More preferably, the method further comprises: (A) providing a first solid discrete region comprising a therapeutically active amount of montelukast, or a pharmaceutically acceptable salt thereof, formulated with one or more pharmaceutically acceptable excipients; (B) providing a second solid discrete region comprising a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, formulated with one or more pharmaceutically acceptable excipients; and (C) combining the first and second solid discrete regions to form the pharmaceutical dosage form.

Preferably, montelukast is present in an amount from about 2 mg to about 10 mg and R411 is present in an amount from about 50 mg to about 400 mg. Preferably, the dosage form is selected from the group consisting of bilayer tablet, a sandwich tablet, a tablet having coated microbeads, and a film coated tablet.

The pharmaceutical dosage forms of the present invention can be prepared according to the examples set out below. The examples are presented for purposes of demonstrating, but not limiting, the preparation of the compounds and compositions of this invention.

### EXAMPLES

In accordance with the present invention, the following examples are provided to illustrate solid pharmaceutical dosage forms, which overcome the incompatibility between R411 and montelukast. A uniform mix of two pre-formulated drugs is illustrated in Example 3 and discrete regions are illustrated in Examples 1, 2, and 4. Combinations of granules, microspheres, microbeads, or minitablets can be mixed in any desired proportion to make either tablets, capsules, sachets, or suspensions.

### Example 1

Bilayer Tablets

Bilayer tablets can be formulated by utilizing twin hopper compression machines. The granulates of each component are prepared individually using pharmaceutically acceptable excipients such as lactose, sucrose, microcrystalline cellulose, stearic acid, hydroxypropylmethylcellulose, polyvinylpyrrolidone, crospovidone, croscarmelose sodium, sodium starch glycolate, dicalcium phosphate, mannitol, sorbitol, silicified microcrystalline cellulose, talc, colloidal silica, stearic acid, or magnesium stearate. The individual granulates can then be compressed together into one unit.

A typical composition of a bilayer tablet has the following composition: a first layer in percentages by weight of the first layer;

| | |
|---|---|
| montelukast | 10% |
| hydroxypropyl cellulose | 4% |
| croscarmellose sodium | 4% |
| lactose hydrous | 56% |
| microcrystalline cellulose | 20% |
| talc | 5% |
| magnesium stearate | 1% |

a second layer in percentages by weight of the second layer;

| | |
|---|---|
| R411 | 50% |
| povidone K30 | 4% |
| crospovidone | 4% |
| lactose hydrous | 26% |
| microcrystalline cellulose | 10% |
| talc | 5% |
| magnesium stearate | 1% |

The required amount of compressed granulates from each layer may then be compressed into a single bilayer tablet. For example, 100 mg of compressed montelukast granulates and 600 mg of compressed R411 granulates may be combined to form a bilayer tablet containing 10 mg of montelukast and 300 mg of R411. Similarly, 50 mg of compressed montelukast granulates and 100 mg of compressed R411 granulates may be combined to form a bilayer tablet containing 5 mg of montelukast and 50 mg of R411.

### Example 2

Sandwich Tablets

Sandwich tablets (or tablets inside tablets) can be prepared by sandwiching a tablet of a montelukast unit into granulates of R411 unit using twin hopper compression machines. The tablet of montelukast is prepared by using standard excipients described above and the granulates of R411 are prepared by conventional granulation techniques using pharmaceutically acceptable excipients. The required amount of montelukast granulates are compressed to form a tablet or a minitablet for lower strengths. These tablets are sandwiched in the granulation of R411 during compression cycles using appropriately equipped compression machines. For example, a 100 mg tablet of montelukast sandwiched between 400 mg of R411 granules will provide 10 mg dose of montelukast and 200 mg dose of R411.

A typical composition of a sandwich tablet has the following composition:
(a) an inner core layer comprising montelukast present in an amount from about 2 mg to about 10 mg; and
(b) an outer surrounding layer comprising R411 present in an amount from about 50 mg to about 400 mg.

Another typical composition of a sandwich tablet has the following composition:
(a) an inner core layer comprising R411 present in an amount from about 50 mg to about 400 mg; and
(b) an outer surrounding layer comprising montelukast present in an amount from about 2 mg to about 10 mg.

### Example 3

Coated Microbeads of Montelukast Mixed with Granulates of R411

Tablets having coated microbeads can be prepared by formulating one of the components, such as montelukast, using either granulation or granulation followed by extrusion-merumerization techniques and coating the component with pharmaceutically acceptable polymers in fluid bed or coating pans in such a proportion that coating provides enough barrier to separate the two active components but does not affect the dissolution behavior of the coated product. The coated microbeads of montelukast can then be mixed with R411 granulates prepared using conventional methods.

A typical composition of a R411 tablet having coated microbeads of montelukast is set out below.

a tablet comprising in percentages by weight of the tablet;

| | |
|---|---|
| R411 | 50% |
| povidone K30 | 4% |
| crospovidone | 4% |
| lactose hydrous | 26% |
| microcrystalline cellulose | 10% |
| talc | 5% |
| magnesium stearate | 1% |

coated microbeads in percentages by weight of the coated microbeads;

| | |
|---|---|
| montelukast | 10% |
| microcrystalline cellulose | 78% |
| hypromellose | 5% |
| croscarmellose sodium | 2% |
| opadry complete coating system | 5% |

In this example, 100 mg of montelukast microbeads and 400 mg of R411 granulates are compressed into tablets or filled into capsules to provide a fixed combination containing 10 mg of montelukast and 200 mg of R411.

A typical manufacturing procedure for tablets having coated microbeads is set out below.

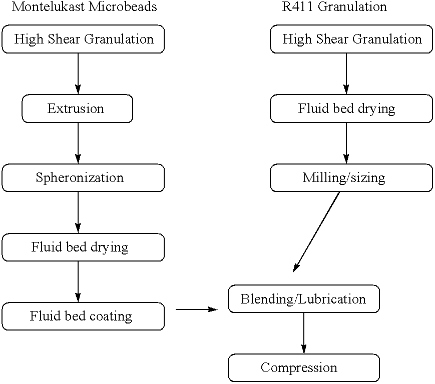

### Example 4

Montelukast Layered Tablets

Film-coated tablets can be prepared by incorporating montelukast in a film-coating layer. Tablets of R411 are prepared by conventional manufacturing processes such as granulation, milling, blending lubricating, and compressing. The required dose of montelukast is dissolved in a coating dispersion usually consisting of film forming agents such as hypromellose, polyvinyl alcohol, starch or ethylcellulose along with a gliding agent such as talc, colorant and plasticizer (triacetin, dibutylsebacate, polyethylene glycol) dispersed in water. The required amount of montelukast film coating is then applied over the R411 kernel tablet either in a pan coater or fluidbed coater to deposit the specific amount of montelukast onto the R411 kernels.

A typical composition of a film-coated tablet comprises: a tablet comprising in percentages by weight of the film coated tablet;

| | |
|---|---|
| R411 | 45% |
| povidone K30 | 4.00% |
| crospovidone | 4.00% |
| lactose hydrous | 24.75% |
| microcrystalline cellulose | 10.00% |
| talc | 5.00% |
| magnesium stearate | 1.00% |

a film coating covering the tablet comprising in percentages by weight of the film coated tablet;

| | |
|---|---|
| montelukast | 2.25% |
| opadry complete coating system | 4.00% |

In this example, R411 granulation is compressed into a tablet to contain 200 mg R411. The compressed tablets are then film-coated with Opadry complete coating system that contains dissolved/dispersed montelukast. A 445 mg film-coated tablet as shown in this example delivers 10 mg montelukast and 200 mg of R411. The film-coat may comprise of any other film-forming polymer such as Plasdone S630, ethycellulose, polyvinyl acetate, polyvinyl alcohol, Eudragit, such as methylmethacrylates with or without plasticizers (triacetin, triethyl citrate, dibutylsebacate, polyethylene glycol) ets. And the coating system can be dispersed in aqueous or non-aqueous media. The aqueous media may be appropriately buffered to achieve the maximum solubility and stability of montelukast.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A solid pharmaceutical dosage form for oral administration comprising a therapeutically active amount of montelukast, or a pharmaceutically acceptable salt thereof, a therapeutically effective amount of N-(2-chloro-6-methylbenzoyl)-4-[(2,6-dichlorobenzoyl) amino]-L-phenylalanine-2-(diethylamino)ethyl ester, or a pharmaceutically acceptable salt thereof, and one or more pharmaceutically acceptable excipients.

2. The dosage form according to claim 1, comprising a combination of two pre-formulated pharmaceutical compositions, wherein a first composition of the two compositions comprises a therapeutically active amount of montelukast, or a pharmaceutically acceptable salt thereof, which first composition is formulated with one or more pharmaceutically acceptable excipients; and a second composition of the two compositions comprises a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, which second composition is formulated with one or more pharmaceutically acceptable excipients.

3. The dosage form according to claim 2, wherein the two pre-formulated compositions are combined as two discrete regions in a single dosage form.

4. The pharmaceutical composition according to claim 1, wherein montelukast is present in an amount from about 2 mg to about 10 mg.

5. The pharmaceutical composition according to claim 1, wherein N-(2-chloro-6-methylbenzoyl) -4-[(2,6-dichlorobenzoyl)amino]-L-phenylalanine-2-(diethylamino)ethyl ester is present in an amount from about 50 mg to about 400 mg.

6. The pharmaceutical composition according to claim 3, wherein the dosage form is selected from the group consisting of a bilayer tablet, a sandwich tablet, a tablet having coated microbeads, and a film coated tablet.

7. The pharmaceutical composition according to claim 6, wherein the bilayer tablet comprises:
(a) a first layer comprising montelukast present in an amount from about 2 mg to about 10 mg; and
(b) a second layer comprising N-(2-chloro-6-methylbenzoyl)-4-[(2,6-dichlorobenzoyl) amino]-L-phenylalanine-2-(diethylamino)ethyl ester present in an amount from about 50 mg to about 400 mg.

8. The pharmaceutical composition according to claim 7, wherein the bilayer tablet has the following composition:
(a) a first layer in percentages by weight of the first layer;

| | |
|---|---|
| montelukast | 10% |
| hydroxypropyl cellulose | 4% |
| croscarmellose sodium | 4% |
| lactose hydrous | 56% |
| microcrystalline cellulose | 20% |
| talc | 5% |
| magnesium stearate | 1% |

(b) a second layer in percentages by weight of the second layer;

| | |
|---|---|
| N-(2-chloro-6-methylbenzoyl)-4-[(2,6-dichlorobenzoyl)amino]- | 50% |
| L-phenylalanine-2-(diethylamino)ethyl ester | |
| povidone K30 | 4% |
| crospovidone | 4% |
| lactose hydrous | 26% |
| microcrystalline cellulose | 10% |
| talc | 5% |
| magnesium stearate | 1% |

9. The pharmaceutical composition according to claim 6, wherein the sandwich tablet comprises:
(a) an inner core layer comprising montelukast present in an amount from about 2 mg to about 10 mg; and
(b) an outer surrounding layer comprising N-(2-chloro-6-methylbenzoyl)-4-[(2,6-dichlorobenzoyl) amino]-L-phenylalanine-2-(diethylamino)ethyl ester present in an amount from about 50 mg to about 400 mg.

10. The pharmaceutical composition according to claim 6, wherein the sandwich tablet comprises:
(a) an inner core layer comprising N-(2-chloro-6-methylbenzoyl)-4-[(2,6-dichlorobenzoyl) amino]-L-phenylalanine-2-(diethylamino)ethyl ester present in an amount from about 50 mg to about 400 mg; and
(b) an outer surrounding layer comprising montelukast present in an amount from about 2 mg to about 10 mg.

11. The pharmaceutical composition according to claim 6, wherein the tablet having coated microbeads comprises:
(a) a tablet comprising N-(2-chloro-6-methylbenzoyl)-4-[(2,6-dichlorobenzoyl) amino]-L-phenylalanine-2-(diethylamino)ethyl ester present in an amount from about 50 mg to about 400 mg; and
(b) coated microbeads dispersed throughout the tablet comprising montelukast present in an amount from about 2 mg to about 10 mg.

12. The pharmaceutical composition according to claim 6, wherein the tablet having coated microbeads comprises:
(a) a tablet comprising montelukast present in an amount from about 2 mg to about 10 mg; and
(b) coated microbeads dispersed throughout the tablet comprising N-(2-chloro-6-methylbenzoyl)-4-[(2,6-dichlorobenzoyl)amino]-L-phenylalanine-2-(diethylamino)ethyl ester present in an amount from about 50 mg to about 400 mg.

13. The pharmaceutical composition according to claim 11, wherein the tablet having coated microbeads comprises:
(a) a tablet comprising in percentages by weight of the tablet;

| | |
|---|---|
| N-(2-chloro-6-methylbenzoyl)-4-[(2,6-dichlorobenzoyl)amino]- | 50% |
| L-phenylalanine-2-(diethylamino)ethyl ester | |
| povidone K30 | 4% |
| crospovidone | 4% |
| lactose hydrous | 26% |
| microcrystalline cellulose | 10% |
| talc | 5% |
| magnesium stearate | 1% |

(b) coated microbeads in percentages by weight of the coated microbeads;

| | |
|---|---|
| montelukast | 10% |
| microcrystalline cellulose | 78% |
| hypromellose | 5% |
| croscarmellose sodium | 2% |
| opadry complete coating system | 5% |

14. The pharmaceutical composition according to claim 6, wherein the film coated tablet comprises:
(a) a tablet comprising N-(2-chloro-6-methylbenzoyl)-4-[(2,6-dichlorobenzoyl) amino]-L-phenylalanine-2-(diethylamino)ethyl ester present in an amount from about 50 mg to about 400 mg; and
(b) a film coating covering the tablet comprising montelukast present in an amount from about 2 mg to about 10 mg.

15. The pharmaceutical composition according to claim 6, wherein the film coated tablet comprises:
(a) a tablet comprising montelukast present in an amount from about 2 mg to about 10 mg; and
(b) a film coating covering the tablet comprising N-(2-chloro-6-methylbenzoyl)-4-[(2,6-dichlorobenzoyl) amino]-L-phenylalanine-2-(diethylamino)ethyl ester present in an amount from about 50 mg to about 400 mg.

16. The pharmaceutical composition according to claim 14, wherein the film coated tablet comprises:
(a) a tablet comprising in percentages by weight of the film coated tablet;

| | |
|---|---|
| N-(2-chloro-6-methylbenzoyl)-4-[(2,6- | 45% |
| dichlorobenzoyl)amino]-L-phenylalanine-2- | |
| (diethylamino)ethyl ester | |
| povidone K30 | 4.00% |
| crospovidone | 4.00% |
| lactose hydrous | 24.75% |
| microcrystalline cellulose | 10.00% |
| talc | 5.00% |
| magnesium stearate | 1.00% |

(b) a film coating covering the tablet comprising in percentages by weight of the film coated tablet;

| | |
|---|---|
| montelukast | 2.25% |
| opadry complete coating system | 4.00% |

17. The pharmaceutical composition according to claim 1, wherein montelukast is in sustained release form and N-(2-chloro-6-methylbenzoyl)-4-[(2,6-dichlorobenzoyl) amino]-L-phenylalanine-2-(diethylamino)ethyl ester is in immediate release form.

18. A method for treating asthma comprising administering to a subject, in need thereof, a solid pharmaceutical dosage form for oral administration comprising a therapeutically active amount of montelukast, or a pharmaceutically acceptable salt thereof, a therapeutically effective amount of N-(2-chloro-6-methylbenzoyl)-4-[(2,6-dichlorobenzoyl) amino]-L-phenylalanine-2-(diethylamino)ethyl ester, or a pharmaceutically acceptable thereof, and one or more pharmaceutically acceptable excipients.

19. The method according to claim 18, wherein the dosage form comprises a combination of two discrete pre-formulated pharmaceutical compositions, wherein a first composition of the two compositions comprises a therapeutically active amount of montelukast, or a pharmaceutically acceptable salt thereof, which first composition is formulated with one or more pharmaceutically acceptable excipients; and a second composition of the two compositions comprises a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, which second composition is formulated with one or more pharmaceutically acceptable excipients.

20. The method according to claim 19, wherein the dosage form comprises two discrete regions, wherein a first region of the two regions comprises a therapeutically effective amount of montelukast, or a pharmaceutically acceptable salt thereof; and a second region of the two regions comprises a therapeutically effective amount of N-(2-chloro-6-methylbenzoyl) 4-[(2,6-dichlorobenzoyl)amino]-L-phenylalanine-2-(diethylamino)ethyl ester, or a pharmaceutically acceptable salt thereof.

21. The method according to claim 18, wherein montelukast is present in an amount from about 2 mg to about 10 mg and N-(2-chloro-6-methylbenzoyl)-4-[(2,6-dichlorobenzoyl) amino]-L-phenylalanine-2-(diethylamino)ethyl ester is present in an amount from about 50 mg to about 400 mg.

22. The method according to claim 20, wherein the dosage form is selected from the group consisting of a bilayer tablet, a sandwich tablet, a tablet having coated microbeads, and a film coated tablet.

23. A method for preparing a solid pharmaceutical dosage form for oral administration comprising admixing a therapeutically active amount of montelukast, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

24. The method according to claim 23, further comprising:
(A) providing a first pre-formulated pharmaceutical composition comprising a therapeutically active amount of montelukast, or a pharmaceutically acceptable salt thereof, formulated with one or more pharmaceutically acceptable excipients;
(B) providing a second pre-formulated pharmaceutical composition comprising a therapeutically effective amount of N-(2-chloro-6-methylbenzoyl)-4-[(2,6-dichlorobenzoyl) amino]-L-phenylalanine-2-(diethylamino)ethyl ester, or a pharmaceutically acceptable salt thereof, formulated with one or more pharmaceutically acceptable excipients; and
(C) combining the first and second solid compositions to form the pharmaceutical dosage form.

25. The method according to claim 24, further comprising:
(A) providing the first pre-formulated composition in a first solid discrete region;
(B) providing the second pre-formulated composition in a second solid discrete region; and
(C) combining the first and second solid discrete regions to form the pharmaceutical dosage form.

26. The method according to claim 23, wherein montelukast is present in an amount from about 2 mg to about 10 mg and N-(2-chloro-6-methylbenzoyl)-4-[(2,6-dichlorobenzoyl) amino]-L-phenylalanine-2-(diethylamino)ethyl ester is present in an amount from about 50 mg to about 400 mg.
